(19) United States
(12) Patent Application Publication
(10) Pub. No.: US 2014/0296173 A1
(43) Pub. Date: Oct. 2, 2014

(54) STABLE NANOCOMPOSITION COMPRISING EPIRUBICIN, PROCESS FOR THE PREPARATION THEREOF, ITS USE AND PHARMACEUTICAL COMPOSITIONS CONTAINING IT

(71) Applicants: János BORBÉLY, Debrecen (HU); Zsuzsanna BERÉNYI, Debrecen (HU); István HAJDÚ, Tiszacsege (HU)

(72) Inventors: János BORBÉLY, Debrecen (HU); Zsuzsanna BERÉNYI, Debrecen (HU); István HAJDÚ, Tiszacsege (HU)

(73) Assignee: BBS NANOTECHNOLOGY LLC., Debrecen (HU)

(21) Appl. No.: 14/228,852

(22) Filed: Mar. 28, 2014

Related U.S. Application Data
(60) Provisional application No. 61/805,956, filed on Mar. 28, 2013.

(51) Int. Cl. A61K 47/36; A61K 47/34; A61K 31/704

(52) U.S. Cl. CPC A61K 47/36; A61K 31/704; A61K 47/34
USPC 514/34

(57) ABSTRACT

A nanoparticulate composition is disclosed for the targeted therapeutic treatment of tumours. The stable self assembled nanocomposition according to the invention comprises (i) a carrier and targeting system comprising an optionally modified polyanion, and optionally a polycation, which may also be modified; at least one targeting agent which is linked to either the polycation/modified polycation or the polyanion/modified polyanion, or both; (ii) an active compound selected from the group of epirubicin and its pharmaceutically acceptable salts, especially hydrochloride; and optionally (iii) at least one complexing agent, metal ion and stabilizer/formulating agent. The invention furthermore relates to a process for the preparation of the above-mentioned composition, the therapeutic uses thereof, and pharmaceutical compositions containing the nanocomposition according to the invention.

This application claims priority to U.S. provisional application Ser. No. 61/805,956, filed Mar. 28, 2013, the entire disclosure of which is hereby incorporated by reference herein.

### FIELD OF THE INVENTION

The present invention relates to a nanoparticulate composition for the targeted therapeutic treatment of tumours. The stable self assembled nanocomposition according to the invention comprises (i) a carrier and targeting system comprising an optionally modified polyanion, and optionally a polycation, which may also be modified; at least one targeting agent which is linked to either the polycation/modified polycation or the polyanion/modified polyanion, or both or to the surface of the nanoparticle; (ii) an active compound selected from the group of epirubicin and its pharmaceutically acceptable salts, especially hydrochloride; and optionally (iii) at least one complexing agent, a metal ion a stabilizer/formulating agent or a PEGylating agent. The present invention furthermore relates to a process for the preparation of the above-mentioned composition, the therapeutic uses thereof, and pharmaceutical compositions containing the nanocomposition according to the invention.

### BACKGROUND OF THE INVENTION

Epirubicin(8R,10S)-10-((2S,4S,5R,6S)-4-amino-5-hydroxy-6-methyltetrahydro-2H-pyran-2-yl)-6,8,11-trihydroxy-8-(2-hydroxyacetyl)-1-methoxy-7,8,9,10-tetrahydrotetracene-5,12-dione, the compound according to Formula I, is a drug used in cancer chemotherapy, often in its hydrochloride salt form.

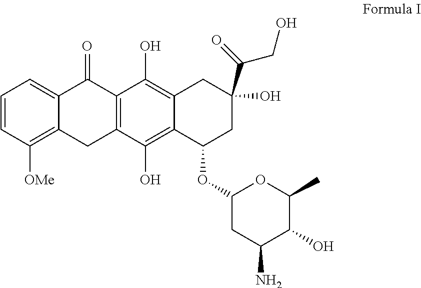

Epirubicin (EPIR) is an anthracycline drug used for chemotherapy. It can be used in combination with other medications to treat breast cancer in patients who have had surgery to remove the tumor. It is marketed by Pfizer under the trade name Ellence in the US and Pharmorubicin or Epirubicin Ebewe elsewhere.

Similarly to other anthracyclines, epirubicin acts by intercalating DNA strands. Intercalation results in complex formation which inhibits DNA and RNA synthesis. It also triggers DNA cleavage by topoisomerase II, resulting in mechanisms that lead to cell death. Binding to cell membranes and plasma proteins may be involved in the compounds cytotoxic effects. Epirubicin also generates free radicals that cause cell and DNA damage.

Acute adverse effects of epirubicin can include nausea, mucositis, vomiting, fatigue and congestive heart failure. It can also cause leukopenia (a decrease in white blood cells), as well as complete alopecia (hair loss). One large retrospective study reported a significantly increased risk of CHF (congestive heart failure) in patients who received cumulative doses greater than 950 mg/m2. The study also found that previous irradiation against the heart leads to an increased risk of developing CHF with an accelerated course to death. This indicates an additive cardiotoxic effect with the use of irradiation and epirubicin. Other serious drug-related cardiovascular adverse events that occurred during clinical trials have included ventricular tachycardia, AV (atrioventricular) block, bundle branch block, bradycardia and thromboembolism. Postmarketing side effects have included arterial embolism, thrombophlebitis, and phlebitis.

Epirubicin is favoured over doxorubicin, the most popular anthracycline, in some chemotherapy regimens as it appears to cause fewer side-effects. Epirubicin has a different spatial orientation of the hydroxyl group at the 4′ carbon of the sugar—it has the opposite chirality—which may account for its faster elimination and reduced toxicity. Epirubicin is primarily used against breast and ovarian cancer, gastric cancer, lung cancer and lymphomas.

### DESCRIPTION OF THE STATE OF THE ART

The problem to be solved in a great number of the chemotherapeutic treatments is the non-specific effect, which means that the chemotherapeutics used is also incorporated in the sane cells and tissues, causing their death. As it can be seen above, the adverse effects of epirubicin cause a limiting factor for the dosing regimen. There is an unmet need to find a composition comprising a carrier and targeting system, which delivers the active compound specifically to the tumour cells, thereby reducing the dose needed, and accordingly, the adverse effects on the intact tissues.

A number of attempts have been made to find a composition which satisfies the above need. U.S. Pat. No. 7,976,825 discloses a macromolecular contrast agent for magnetic resonance imaging. Biomolecules and their modified derivatives form stable complexes with paramagnetic ions thus increasing the molecular relaxivity of carriers. The synthesis of biomolecular based nanodevices for targeted delivery of MRI contrast agents is also described. Nanoparticles have been constructed by self-assembling of chitosan as polycation and poly-gamma glutamic acids as polyanion. Nanoparticles are capable of Gd-ion uptake forming a particle with suitable molecular relaxivity. There is no active agent and therapeutic use disclosed in U.S. Pat. No. 7,976,825.

U.S. Pat. No. 8,007,768 relates to a pharmaceutical composition of the nanoparticles composed of chitosan, a negatively charged substrate, a transition metal ion, and at least one bioactive agent for drug delivery. The nanoparticles are characterized with a positive surface charge configured for promoting enhanced permeability for bioactive agent delivery. The pharmaceutical composition consists of a shell portion that is dominated by positively charged chitosan and a core portion, wherein the core portion consists of the positively charged chitosan, a transition metal ion, one negatively charged substrate, at least one bioactive agent loaded within the nanoparticles, and optionally a zero-charge compound. The composition may contain at least one bioactive agent selected from the group of exendin-4, GLP-1, GLP-1 analog, insulin or insulin analog. Epirubicin is not mentioned among the possible active agents.

WO2009097570 relates to a chemotherapeutic composition configured for subcutaneous administration for preferential intralymphatic accumulation while also providing a therapeutic systemic concentration that is not toxic. The composition can include a pharmaceutically acceptable carrier, and a nanoconjugate configured for preferential intralymphatic accumulation after subcutaneous administration. The nanoconjugate can include a nanocarrier configured for preferential intralymphatic accumulation after subcutaneous or interstitial administration, and a plurality of chemotherapeutic agents coupled to the nanocarrier. The nanoconjugate can have a dimension of about 10 nm to about 50 nm. The nanocarrier can be a hyaluronan polymer of about 3 kDa to about 50 kDa. The chemotherapeutic agent coupled to the nanocarrier via a biodegradable linker can be epirubicin among others. The composition disclosed in the above-mentioned prior art document has a different structure from that of our invention, using different components.

US2006073210 relates to a method of enhancing intestinal or blood brain paracellular transport configured for delivering at least one bioactive agent in a patient comprising administering nanoparticles composed of [gamma]-PGA and chitosan. The administration of the nanoparticles takes place orally. The chitosan is a low molecular weight chitosan (50 kDa) and dominates on a surface of said nanoparticles. The surface of said nanoparticles is characterized by a positive surface charge. The nanoparticles have a mean particle size between about 50 and 400 nanometers and are formed via a simple and mild ionic-gelation method. The nanoparticles are loaded with a therapeutically effective amount of at least one bioactive agent. In the above-mentioned prior art document epirubicin is not mentioned as possible therapeutically active agent. Furthermore, though the composition may enhance the penetration of the blood brain carrier, targeting of the therapeutics has not been solved by the invention.

WO2006042146 relates to conjugates comprising a nanocarrier, a therapeutic agent or imaging agent and a targeting agent, wherein the nanocarrier comprises a nanoparticle, an organic polymer, or both. The organic polymer can comprise a polyamino acid, a polysaccharide, or combinations thereof and the organic polymer can be a polyionic polymer. The use of hyaluronic acid, polyglutamic acid, chitosan, copolymers thereof or combinations thereof is described as the organic polymer. Nanocarriers made of paramagnetic metal ions are described. The use of epirubicin is not mentioned in the prior art document.

The state of the art failed to solve the above-mentioned problem that is the reduction of the adverse effects of epirubicin through the decrease of the incorporated active agent by its targeted delivery. There is an unsatisfied need to provide for a stable composition for the targeted therapeutic treatment of tumours using epirubicin. We performed systematic research in the field and, as a result of our surprising findings, completed our invention.

### BRIEF DESCRIPTION OF THE DRAWINGS

FIG. 1: Size distribution by volume

FIG. 2 a-b: HeLa and A2780 measured by Real Time Analyser (Roche)

FIG. 3 a-k: MTT results

### DETAILED DESCRIPTION OF THE INVENTION

We have surprisingly found that a stable, self assembling nanocomposition may be prepared by using a polycation together with a polyanion when preparing the carrier of the pharmaceutically active agent. The nanocarrier system according to the present invention consists of at least four components: a polycation, a polyanion, an active agent, which is epirubicin or a derivative thereof, especially its hydrochloride salt, and a targeting molecule, which may be linked to the polycation, the polyanion or both, or to the surface of the nanoparticle. The composition may additionally contain a complexing agent bound covalently to the polycation and a stabilizer/formulating agent, or a PEGylating agent, though these are not necessarily included the composition. The formation of the nanoparticles takes place by the self assembling of the polyelectrolites.

Accordingly, in its first aspect the invention relates to a stable self assembled composition comprising
(i) a carrier and targeting system comprising an optionally modified polyanion, and optionally a polycation, which may also be modified; at least one targeting agent which is linked to either the polycation/modified polycation or the polyanion/modified polyanion, or both, or to the surface of the nanoparticle;
(ii) an active compound selected from the group of epirubicin and its pharmaceutically acceptable salts, especially hydrochloride; and optionally
(iii) at least one complexing agent, metal ion and stabilizer/formulating agent, or a PEGylating agent

In a preferred embodiment, the biopolymers are water-soluble, biocompatible, biodegradable polyelectrolyte biopolymers.

One of the polyelectrolyte biopolymers is a polycation, a positively charged polymer, which is preferably chitosan (CH) or any of its derivatives. E.g. in the composition according to the invention the polycation may be chitosan, the modified polycation may be selected from the derivatives of chitosan, especially chitosan-EDTA, chitosan-DOTA, chitosan-DTPA, chitosan-FA, chitosan-LHRH, chitosan-RGD CH-EDTA_FA, CH-FA-EDTA, CH-DOTA-FA, CH-FA-DOTA, CH-DTPA-FA, CH-FA-DTPA, however, they are not limited thereto.

The other type of the polyelectrolyte biopolymers is a polyanion, a negatively charged biopolymer. Preferably the polyanion is selected from the group of poly-gamma-glutamic acid (PGA), polyacrylic acid (PAA), hyaluronic acid (HA), alginic acid (ALG), and the modified derivatives thereof. The modified polyanion is selected from the derivatives of PGA, especially PGA-EPIR, PGA-FA, PGA-FA-EPIR, PGA-LHRH, PGA-RGD.

The derivatives of biopolymers can be their cross-linked nanosystems, biopolymer-complexone conjugates, targeting agent—biopolymer product or other grafted derivatives resulted in modifications of biopolymers with other molecules, e.g. polyethylene glycol (PEG) oligomers.

Preferably the complexing agent is selected from the group of diethylenetriaminepentaacetic acid (DTPA), 1,4,7,10-tetracyclododecane-N,—N′,N″,N″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethylether) N,N,N′,N′,-tetraacetic acid (EGTA), 1,4,8,11-tetraazacyclotradecane-N,N′,N″,N′″-tetraacetic acid (TETA), and 1,4,7-triazacyclononane-N,N′,N″-triacetic acid (NOTA), but is not limited to these materials.

Preferably the targeting agent is selected from the group of small molecules, preferably folic acid (FA), peptides, preferably luteinsing hormone releasing hormone (LHRH), arginin-glycin-aspartate amino acid sequence (RGD), a monoclonal antibody, preferably Transtuzumab.

The formulating agent is selected from the group of glucose, physiological salt solution, PBS. or any of their combination thereof.

The metal ion is selected from the group of calcium, magnesium, copper, gadolinium, gallium.

In a preferred embodiment, the drug molecules are ionically or covalently attached to the bioanion or its derivatives via their functional groups. In case of covalent conjugation, water-soluble carbodiimide, as coupling agent is used to make stable amide bonds between the drug molecules and the biopolymers via their carboxyl and amino functional groups in aqueous media.

As used in the present invention the abbreviations below have the following meanings:
PGA means poly-gamma-glutamine acid
PAA means polyacrylic acid
HA means hyaluronic acid
ALG means alginic acid
CH means chitosan
FA means folic acid
LHRH means luteinsing hormone releasing hormone
RGD means arginin-glycin-aspartate amino acid sequence
EPIR means epirubicin
DTPA means diethylene-triamine-pentaacetic acid
DOTA means 1,4,7,10-tetracyclododecane-N,—N′,N″,N′″-tetraacetic acid
EDTA means ethylene-diaminetetraacetic acid
DO3A means 1,4,7,10-tetraazacyclododecane-N,N′,N″-triacetic acid
CHTA means 1,2-diaminocyclohexane-N,N,N′,N′-tetraacetic acid
EGTA means ethylene glycol-bis(beta-aminoethylether) N,N,N′,N′,-tetraacetic acid
TETA means 1,4,8,11-tetraazacyclotradecane-N,N′,N″,N′″-tetraacetic acid
NOTA means 1,4,7-triazacyclononane-N,N′,N″-triacetic acid
PGA-FA means poly-gamma-glutamic acid-bound folic acid
PGA-EPIR means poly-gamma-glutamic acid-bound epirubicin
PGA-FA-EPIR means folic acid-PGA-bound epirubicin
PGA-LHRH means poly-gamma-glutamic acid-bound luteinsing hormone releasing hormone
PGA-RGD means poly-gamma-glutamic acid-bound arginin-glycin-aspartate amino acid sequence
PAA-FA means polyacrylic acid-bound folic acid
PAA-LHRH means polyacrylic acid-bound luteinsing hormone releasing hormone
PAA-RGD means polyacrylic acid-bound arginin-glycin-aspartate amino acid sequence
HA-FA means hyaluronic acid-bound folic acid
HA-RGD hyaluronic acid-bound arginin-glycin-aspartate amino acid sequence
HA-LHRH hyaluronic acid-bound luteinsing hormone releasing hormone
ALG-FA means alginic acid-bound folic acid
ALG-LHRH means alginic acid-bound luteinsing hormone releasing hormone
ALG-RGD means alginic acid-bound arginin-glycin-aspartate amino acid sequence CH-EDTA means chitosan-bound ethylene-diaminetetraacetic acid
CH-DOTA means chitosan-bound 1,4,7,10-tetracyclododecane-N,—N′,N″,N′″-tetraacetic acid CH-DTPA means chitosan-bound diethylene-triamine-pentaacetic acid
CH-FA means chitosan-bound folic acid
CH-LHRH means chitosan-bound luteinsing hormone releasing hormone
CH-RGD means chitosan-bound arginin-glycin-aspartate amino acid sequence
CH-EDTA-FA means chitosan-bound ethylene-diaminetetraacetic acid and folic acid
CH-FA-EDTA means chitosan-bound folic acid and ethylene-diaminetetraacetic acid
CH-DOTA-FA means chitosan-bound 1,4,7,10-tetracyclododecane-N,—N′,N″,N′″-tetraacetic acid and folic acid
CH-FA-DOTA means chitosan-bound folic acid and 1,4,7,10-tetracyclododecane-N,—N′,N″,N′″-tetraacetic acid
CH-DTPA-FA means chitosan-bound diethylene-triamine-pentaacetic acid and folic acid
CH-FA-DTPA means chitosan-bound folic acid and diethylene-triamine-pentaacetic acidEDC*HCl means (1-ethyl-3-(3-dimethylaminopropyl)-carbodiimide methiodide)
DMSO means dimethyl-sulphoxide
NaOH means sodium-hydroxide
PA means polyanion
PC means polycation
PD-EPIR means epirubicin loaded polymer
NP means nanoparticle
NP-EPIR means epirubicin loaded nanoparticle
HOBt means 1-hydroxybenzotriazole hydrate
TEA means tryethylamine
PEG means polyethylene glycol
MeO-PEG-NH₂ means methoxy polyethylene glycol amine
FA-PEG-NH₂ means folic acid polyethylene glycol amine
PGA-CA means poly-gamma-glutamic acid bound citric acid
FA-PEG means pegylated folic acid
PGA-PEG-FA means poly-gamma-glutamic acid bound pegylated folic acid
PGA-PEG-FA-EPIR means epirubicin loaded PGA-PEG-FA
NP-PEG means pegylated nanoparticles
NP-EPIR-PEG means epirubicin loaded pegylated nanoparticles
NP-PEG-FA means nanoparticles bound FA-PEG.
NP-EPIR-PEG-FA means epirubicin loaded nanoparticles bound FA-PEG

A preferred self-assembled composition according to the present invention is characterized by any or more of the following features:
(i) the average size of the nanoparticles in swollen state is in the range between 30 to 500 nm, preferably 60 to 200 nm, more preferably about 80 to 120 nm;
(ii) the proportion of the polycation to the polyanion is about 1:20 to 20:1 based on the weight of the agents, preferably about 1:2;
(iii) the polyanion has a pH of 7.5 to 10; a molecular weight of 10 000 Da to 1.5 MDa and a concentration of 0.01 to 2 mg/ml, preferably 0.3 mg/ml;
(iv) the polycation has a pH of 3.5 to 6; a molecular weight of 60 to 320 kDa and a concentration of 0.01 to 2 mg/ml, preferably 0.3 mg/ml.

In its second aspect the present invention relates to a process for the preparation of the above mentioned composition according to the invention, characterized in that it comprises the steps of
(i) a targeting agent is bound covalently to the polyanion;
(ii) the active agent is bound covalently or by an ionic bond to the polyanion;
(iii) the polycation and the polyanion are contacted with each other, preferably in a ratio of 1:20 to 20:1, more preferably about 1:2 based on the weight of the agents, thus are reacted with each other to self-assemble;
(iv) optionally the other components are added to the reaction mixture.

In a preferred embodiment the polyanion used in the process according to the invention has a pH of 7.5 to 10; a molecular weight of 10 000 Da to 1.5 MDa and a concentration of 0.01 to 2 mg/ml; and the polycation used has a pH of 3.5 to 6; a molecular weight of 60 to 320 kDa and a concentration of 0.01 to 2 mg/ml.

In a further preferred embodiment the other components that are added to the reaction mixture are complexing agents which are bound to the polication.

In a further preferred embodiment of the invention the nanoparticles are formed via an ionotropic gelation, they contain one polyanion and one polycation and are characterized by negative surface charge.

Prior to the reaction of the polyelectrolites any of them or all of them is/are bound to a targeting agent by a covalent bond, thus the nanoparticles will cumulate in the tumourous cells. Furthermore, an active agent according to the present invention is bound to the polyanion, either by covalent or by ionic bond. It is critical to form such a bond between the active compound and the polyanion, which is likely to be split only when incorporated in the target cell, and so the active compound is being released, inside the target cell.

On reaction of the polycation and the polyanion a self-assembly takes place, contracting the molecule and resulting in a stable nanosystem. The thus formed nanoparticles possess negative surface charge and a narrow range of size distribution, which ensure the uniform physical and chemical characteristics. The resulting composition is a hydrophilic nanosystem, forming stable colloid systems in water.

The nanosystem can be designed to achieve compositions with exactly expected features. The type of the self-assembling biopolymers, the order of admixing of the polycation and the polyanion (or their modified derivatives), the molecular weight, the mass ratio, the concentration and the pH of the polycation and the polyanion (or their modified derivatives) will result in different features (size, surface charge, active agent content, targeting agent content, etc.) of the system. The selection of the above elements may be done by a skilled person, knowing the object without undue experimentation.

Furthermore, the present invention relates to a stable self-assembled composition comprising
(i) a carrier and targeting system comprising an optionally modified polycation, and an optionally modified polyanion; at least one targeting agent which is linked to either the polycation/modified polycation or the polyanion/modified polyanion, or both; or to the surface of the nanoparticle.
(ii) an active compound selected from the group of epirubicin and its pharmaceutically acceptable salts, especially hydrochloride; and optionally
(iii) at least one complexing agent, metal ion and stabilizer/formulating agent, or a PEGylating agent which is obtainable by the above-mentioned process according to the invention.

In its third aspect the invention relates to a pharmaceutical composition comprising the composition according to the invention together with pharmaceutically acceptable auxiliary materials, preferably selected from group of glucose, physiological salt solution, and PBS, or any of their combination thereof. Furthermore, the present invention relates to the use of the composition according to the invention or the pharmaceutical composition according the invention for the preparation of a medicament; and the use of the composition or the pharmaceutical composition according to the invention for the treatment of tumours. Finally the invention relates to a method for the treatment of a subject in need for the treatment of tumours, especially human cervical carcinoma (HeLa, KB), human ovary carcinoma (A2780, SK-OV-3, OVCAR-3), human lung adenocarcinoma (A549, H1975), human breast carcinoma (MCF-7, MDA-MB-231), human prostate carcinoma (PC-3, LNCaP), human skin melanoma (HT168-M1/9), human colon adenocarcinoma (HT29), human melanoma (WM983A) and human metastatic melanoma (WM983B) cell lines by administering to the subject an effective amount of the composition or the pharmaceutical composition according to the present invention.

The nanoparticles according to the present invention may be further modified, as follows:
1. PEGylation of the nanoparticle: the prepared nanoparticles may be coated with PEG (poly-ethylene-glycol). With the use of the PEGylated nanoparticles the side effects, e.g. the non-desired accumulation of the nanoparticle in the organs, or the weigh-loss may be decreased;
2 PEG-folic-acidation of the nanoparticle: the prepared nanoparticle is coated with a PEG-chain, which contains folic acid at the end of the chain, thereby better targeting can be achieved;
3 PEG-folic acid association with PGA: the folic acid content of polymers, and thereby nanoparticles can be increased by linking the folic acid to the polimers through a PEG-chain rather than directly. By this method the reaction efficiency may be increased. It is noted that in all three cases a PEG with shorter or longer chain may be of use, e.g. with_{—}750 Da, 2000 Da, 3400 Da, 5000 Da molecular weight;

Accordingly, a further aspect of the present invention is a composition according to the invention, wherein
a) the prepared nanoparticles are further coated with PEG (poly-ethylene-glycol); and/or
b) the prepared nanoparticles are further coated with a PEG-chain, which contains folic acid at the end of the chain; and/or
c) the folic acid content of polymers, and thereby nanoparticles is further increased by linking the folic acid to the polimers through a PEG-chain.

### EXAMPLES

### Preparation of the Formulation According to the Invention

Nanoparticles can be formed by adding polyanion(s) to polycation(s) or the other way round. The addition order of the polyelectrolytes affects the size of the nanoparticles and to a small extent also their surface charge. In both cases the nanoparticle has the structure of a statistical ball, however, significantly smaller particles with narrower size distribution are formed if the polycation (PC) is added to the polyanion (PA).

With higher polymer concentration, the size of the formed nanoparticles is also bigger. This may be avoided by the preparation of the nanoparticles in diluted polymer solution, resulting in smaller size and narrower size distribution. The solution of the so-formed nanoparticles is concentrated afterwards.

Tests of the effectiveness of the compositions according to the invention

The internalization and accumulation of the nanosystem according to the present invention were proved on different cell lines in vitro; the cytotoxicity of the nanosystem was tested by investigating the viability of the cells using the MTT method, on among others human cervical carcinoma (HeLa, KB), human ovary carcinoma (A2780, SK-OV-3, OVCAR-3), human lung adenocarcinoma (A549, H1975), human breast carcinoma (MCF-7, MDA-MB-231), human prostate carcinoma (PC-3, LNCaP), human skin melanoma (HT168-M1/9), human colon adenocarcinoma (HT29), human melanoma (WM983A) and human metastatic melanoma (WM983B) cell line

The drug-loaded nanosystems are stable at pH 7.4, and may be injected intravenously. Based on the blood circulation, the nanoparticles could be transported to the area of interest.

The osmolarity of nanosystem was adjusted to the value of human serum. In a preferred embodiment, the osmolarity was set using formulating agent, selected from the group of glucose, physiological salt solution, PBS or their combination thereof.

The effects of glucose, physiological saline solution, infusion base solutions and different buffers on the size, size distribution and stability of the nanoparticles were investigated.

The xCELLigence RTCA HT Instrument from Roche Applied Science uses gold electrodes at the bottom surface of microplate wells as sensors to which an alternating current is applied. Cells that are grown as adherent monolayers on top of such electrodes influence the alternating current at the electrodes by changing the electrical resistance (impedance). The degree of this change is primarily determined by the number of cells, strength of the cell-cell interactions, interactions of the cells with the microelectrodes and by the overall morphology of the cells. The RTCA Software calculates the Cell Index (CI) as the relative change in measured impedance to represent cell status. The normalized cell index (NCI-plotted on y axis) is the relative cell impedance presented in the percentage of the value at the base-time. NCI shows rate of the surface covered by cells. NCI increases by rise of cell-number or cell-size. For example NCI value in a culture treated with a proliferation inhibitory drug first can increase (because the cell-size grows) and after decreases (because the cell-number reduces)

The MTT test is a colorimetric assay that measures the reduction of yellow 3-(4,5-dimethylthiazol-2-yl)-2,5-diphenyl tetrazolium bromide (MTT) by mitochondrial succinate dehydrogenase. The MTT enters the cells and passes into the mitochondria where it is reduced to an insoluble, coloured (dark purple) formazan product. The cells are then solubilised with an organic solvent (dimethyl sulfoxide) and the released, solubilised formazan reagent is measured spectrophotometrically. Since reduction of MTT can only occur in metabolically active cells the level of activity is a measure of the viability of the cells. This method can therefore be used to measure cytotoxicity, proliferation or activation.

### Cell Lines:

| Cell line | Type of carcinomacell |
|---|---|
| HeLa | Human cervicaladenocarcinomacell line |
| A2780 | Human ovarycarcinoma cell line |
| AD2780 | Human ovarycarcinoma cell line (dox. res.) |
| SK-OV-3 | Human ovary adenocarcinoma cell line |
| A549 | Human lung adenocarcinoma cell line |
| H1975 | Human lung adenocarcinoma cell line |
| MCF-7 | Human breastcarcinoma cell line |
| PC-3 | Human prostatecarcinoma cell line |
| LNCaP | Human prostatecarcinoma cell line |
| KB | Human cervicalcarcinoma cell line |
| HT168-M1/9 | Human skinmelanoma cell line |
| MDA-MB-231 | Human breastcarcinoma cell line |
| HT29 | Human colon adenocarcinoma cell line |
| WM983A | Human melanoma cell line |
| WM983B | Human metastaticmelanoma cell line |
| HaCaT | Human keratinocyte cell line |

### EXAMPLES

### Example 1

### Preparation of Folated Poly-Gamma-Glutamic Acid (γ-PGA)

Folic acid was conjugated via the amino groups to γ-PGA using carbodiimide technique. γ-PGA (m=50 mg) was dissolved in water (V=50 ml) to produce aqueous solution. After the addition of 1-[3-(dimethylamino)propyl]-3-ethylcarbodiimide methiodide (EDC*HCl) (m=22 mg) to the γ-PGA aqueous solution, the reaction mixture was stirred at 4° C. for 30 min. After that, folic acid (m=32 mg in dimethyl sulfoxide, V=10 ml) was added dropwise to the reaction mixture and stirred at room temperature for 24 h. The folated poly-γ-glutamic acid (PGA-FA) was purified with membrane filtration. The reaction may be illustrated by the scheme below.

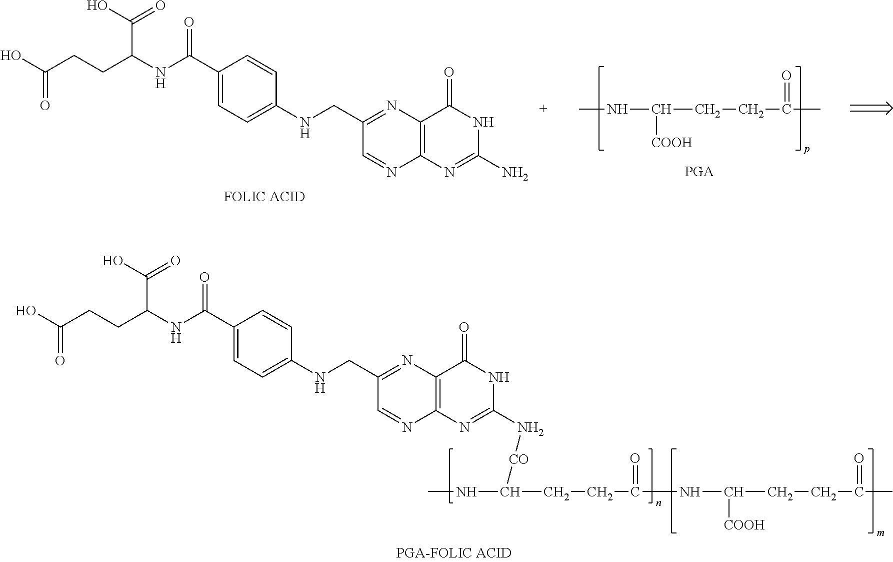

PEG-Folic Acid Association with PGA:

Poly-gamma-glutamic acid (m=300 mg) was solubilized in water (V=300 ml), then HOBt (m=94 mg) was added to the PGA solution. The solution was stirred at 4° C. for 15 minutes, then 1-(3-dimethylaminopropyl)-3-ethylcarbodiimide hydrochloride (EDC*HCl) (m=445 mg in 15 ml water) was added to the solution. The mixture was stirred for 10 minutes while cooling on ice, then folic acid-PEG-amine (NH₂-PEG-NH-FA) ((m=100 mg in 10 ml water) and TEA (m=235 mg) was added to the reaction mixture and stirred at room temperature in the dark for 24 hours. The PGA-FA-PEG was purified with membrane filtration.

### Example 2

### Preparation of Folated Chitosan

A solution of 1-(3-dimethylaminopropyl)-3-ethylcarbodiimide hydrochloride (EDC*HCl) and FA in anhydrous DMSO was prepared and stirred at room temperature until FA was well dissolved (1 h). Chitosan was dissolved in 0.1 M hydrochloric acid, to produce a solution with a concentration of 1 mg/ml, and then adjusted to pH 5.5 with 0.10 M sodium hydroxide solution. After the dropwise addition of EDC*HCl (m=5.1 mg in 1 ml distilled water) to the chitosan solution (V=20 ml), the reaction mixture was stirred for 10 min. Then folic acid (m=8.5 mg in dimethyl sulfoxide, V=1 ml) was added to the reaction mixture. The resulting mixture was stirred at room temperature in the dark for 24 h. It was brought to pH 9.0 by drop wise addition of diluted aqueous NaOH and was washed three times with aqueous NaOH, and once with distilled water. The polymer was isolated by lyophilization

### Example 3

### Preparation of Chitosan-DTPA Conjugate

Chitosan (m=15 mg) was solubilized in distilled water (V=15 ml); its dissolution was facilitated by dropwise addition of 0.1 M HCl solution. After the dissolution, the pH of chitosan solution was adjusted to 5.0. After the dropwise addition of DTPA aqueous solution (m=7 mg, V=2 ml, pH=5.0), the reaction mixture was stirred at room temperature for 30 min, and at 4° C. for 15 min. after that, EDC*HCl (m=5.35 mg, V=2 ml distilled water) was added dropwise to the reaction mixture and stirred at 4° C. for 4 h, then at room temperature for 20 h. The chitosan-DTPA conjugate (CH-DTPA) was purified by dialysis, or with membrane filtration.

### Example 4

### Preparation of Chitosan-EDTA Conjugate

Deacylated chitosan (m=15 mg) was solubilized in distilled water (V=15 ml). After the dissolution, the pH of chitosan solution was adjusted to 4.8. After the dropwise addition of EDTA aqueous solution (m=7.8 mg, V=7.8 ml, pH=6.3), the reaction mixture was stirred at room temperature for 30 min, and at 4° C. for 15 min. Then, EDC*HCl (m=7.9 mg, V=1 ml in distilled water), was added dropwise to the reaction mixture and stirred at 4° C. for 4 h, then at room temperature for 20 h. The chitosan-EDTA conjugate (CH-EDTA) was purified by dialysis.

### Example 5

### Preparation of Epirubicin Loaded Poly-Gamma-Glutamic Acid—Ionically Bound

Poly-gamma-glutamic acid (m=5.0 mg) was dissolved in distilled water (V=10 ml) and then adjusted to pH 9.5. Epirubicin (EPIR) solution (V=400 μl) with a concentration of c=2 mg/ml was added to the PGA solution and the reaction was stirred for 24 h at room temperature. The epirubicin-loaded PGA was purified by dialysis, or with membrane filtration.

### Example 6

### Preparation of Epirubicin Loaded Folated-Poly-Gamma-Glutamic Acid—Covalently Bound

Folated—PGA (m=5.0 mg) was dissolved in distilled water (V=10 ml) and then adjusted to pH 5.5. m=0.4 mg of water soluble EDC*HCl was dissolved in distilled water (V=4000). The PGA solution was stirred for 10 min at 4° C. EDC*HCl solution was added dropwise to the PGA solution and was stirred for 40 min at 4° C. and 30 min at room temperature. After that EPIR solution (c=2 mg/ml, V=400 μl) was added dropwise to the mixture, and the reaction was stirred for 24 h at room temperature. The EPIR-loaded PGA was purified by dialysis or with membrane filtration. The reaction may be illustrated by the scheme below.

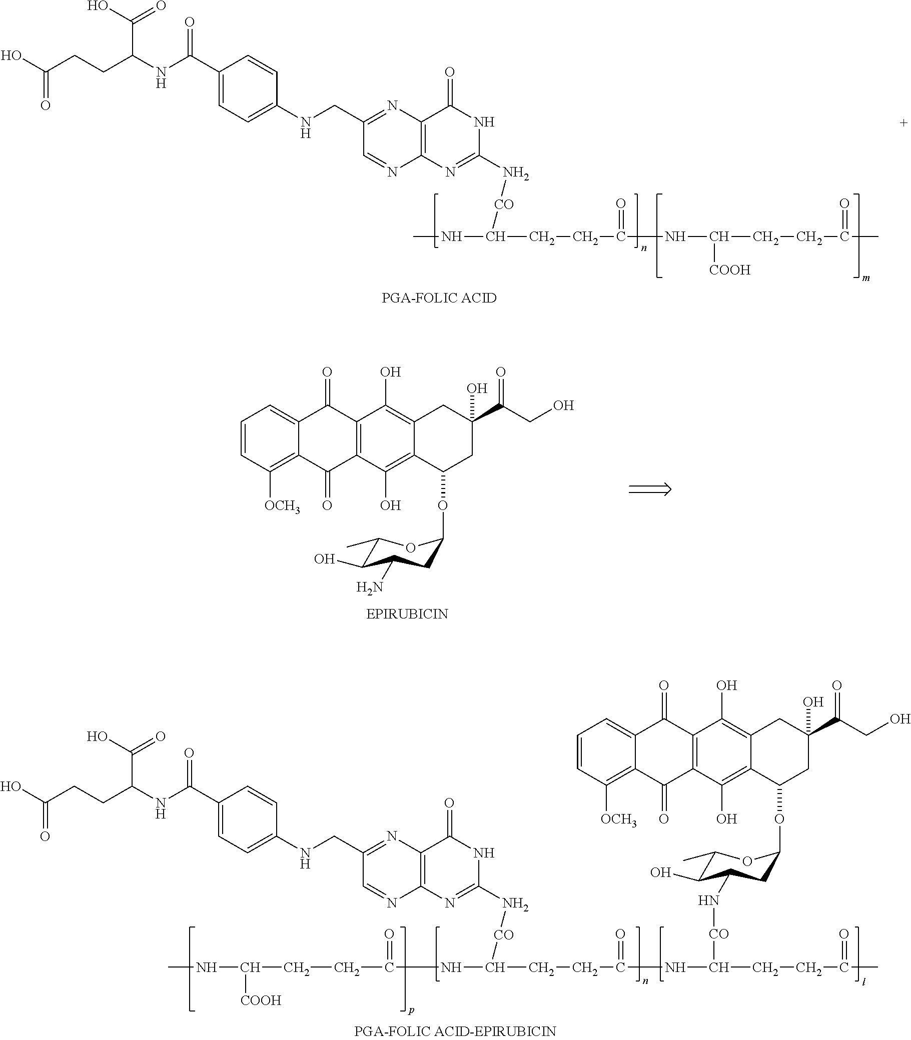

### Example 7

### Preparation of Epirubicin Loaded PEG-Folated-Poly-Gamma-Glutamic Acid (PGA-PEG-FA-EPIR)—Covalently Bound, with HOBt

PEG-folated-poly-gamma-glutamic acid (m=20.0 mg) was dissolved in distilled water (V=40 ml). Epirubicin (V=4500 μl c=2 mg/ml) was added to the PGA solution by consecutive dropwise addition. The reaction mixture was stirred at room temperature for 40 minutes, then for 15 minutes at 4° C. Meanwhile EDC*HCl (m=7.56 mg) was dissolved in V=2 ml distilled water and mixed with m=3.36 mg HOBt dissolved in V=2 ml distilled water to produce a mixture, which was then stirred for 30 minutes at room temperature. After the reaction mixture was stirred on ice for 15 minutes the EDC*HCl-HOBT mixture was added to the reaction by dropwise addition and stirred at 4° C. for 4 hours, then at room temperature for 20 hours. The PGA-PEG-FA-EPIR was purified with membrane filtration.

### Example 8

### Preparation of Targeting, Epirubicin Loaded, Self-Assembled Poly-Gamma-Glutamic Acid/Chitosan Nanoparticles

Folated PGA solution (c=0.3 mg/ml) and EPIR-loaded PGA solution (c=0.3 mg/ml) were mixed at a ratio of 1:1. The pH of mixture was adjusted to 9.5. Chitosan was dissolved in water ((c=0.3 mg/ml). Its dissolution was facilitated by dropwise addition of 0.1 M HCl solution and the pH was adjusted to 4.0. Chitosan solution (V=1 ml) was added to the PGA mixture (V=2 ml), and was stirred at room temperature for 15 min. It is noted that the nanosystem can be prepared by a number of methods, the scheme is only one example for the preparation of the three phase system.

### Example 9

### Preparation of Targeting, Epirubicin Loaded, Self-Assembled Poly-Gamma-Glutamic Acid/Chitosan Nanoparticles

PEG-folated epirubicin loaded PGA (PGA-PEG-FA-EPIR) solution was prepared (c_{polymer}=0.3 mg/ml.) The pH of the solution was adjusted to 8.0. CH-EDTA was dissolved in aqueous medium (c_{polymer}=0.3 mg/ml), and the pH was adjusted to 4.0. CH-EDTA solution (V=1 ml) was added dropwise to the PGA-PEG-FA-EPIR solution (V=2 ml) under continuous stirring. It is noted that the nanosystem can be prepared by a number of methods, the scheme is only one example for the preparation of the two phase system.

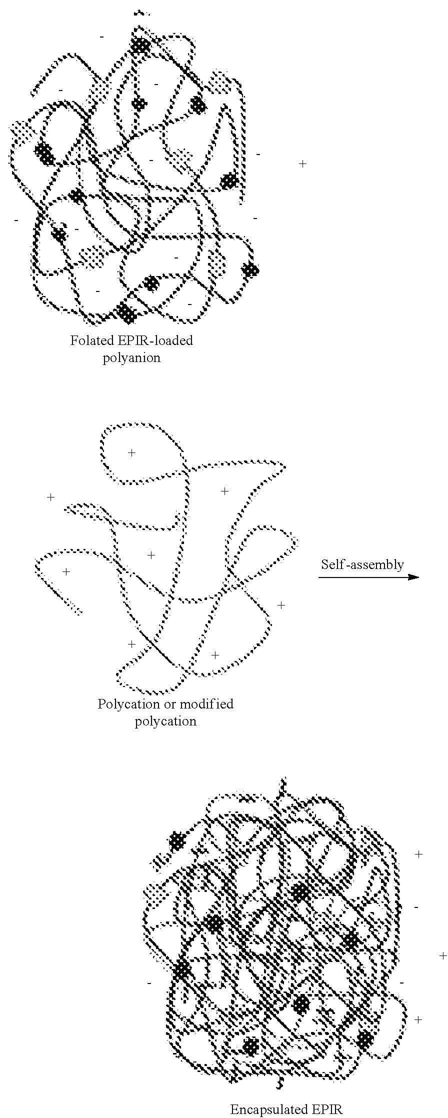

### Example 10

### Preparation of NP-EPIR-PEG (Pegylation with MeO-PEG-NH₂ 2000 Da)

15.4 mg MeO-PEG-NH₂ was added drop wise to 50 ml epirubicin loaded NP (c_{polymer}=0.3 mg/ml) and the solution was stirred for 30 minutes at room temperature, then for 15 minutes at 4° C. 3.4 mg EDC*HCl was dissolved in 1 ml distillated water and mixed with 1.56 mg HOBt dissolved in 1 ml distillated water to produce a mixture. The mixture was then added to the reaction. The reaction was stirred at 4° C. for 4 hours then room temperature for 20 hours. The pegylated NP was purified with membrane filtration.

### Example 11

### Preparation of NP-EPIR-PEG-FA (Pegylation with FA-PEG-NH₂ 2000 Da)

15.4 mg FA-PEG-NH₂ was added drop wise to 50 ml epirubicin loaded NP (c_{polymer}=0.3 mg/ml) and the solution was stirred for 30 minutes at room temperature, then for 15 minutes at 4° C. 3.4 mg EDC*HCl was dissolved in 1 ml distillated water and mixed with 1.56 mg HOBt dissolved in 1 ml distillated water to produce a mixture. The mixture was then added to the reaction. The reaction was stirred at 4° C. for 4 hours then room temperature for 20 hours. The pegylated NP was purified with membrane filtration.

### Example 12

### Characterization of Self-Assembled, Drug-Laded Nanoparticles

The hydrodynamic size and size distribution of particles was measured using a dynamic light scattering (DLS) technique with a Zetasizer Nano ZS (Malvern Instruments Ltd., Grovewood, Worcestershire, UK). This system is equipped with a 4 mW helium/neon laser with a wavelength of 633 nm and measures the particle size with the noninvasive backscattering technology at a detection angle of 173°. Particle size measurements were performed using a particle-sizing cell in the automatic mode. The mean hydrodynamic diameter was calculated from the autocorrelation function of the intensity of light scattered from the particles. Electrokinetic mobility of the nanoparticles was measured in folded capillary cell (Malvern) with a Zetasizer Nano ZS apparatus.

### Example 13

### Cellular Uptake of Self-Assembled, Drug-Loaded Nanoparticles

Internalization and selectivity of nanoparticulates was investigated in cultured human cancer cells overexpressing folate receptors by using confocal microscopy and flow cytometry. The samples were imaged on an Olympus FluoView 1000 confocal microscope. Excitation was performed by using the 488 nm line of an Ar ion laser (detection: 500-550 nm) and the 543 nm line of a HeNe laser (detection: 560-610 nm) to image Alexa 488 and Alexa 546 respectively. Images were analyzed using Olympus FV10-ASW 1.5 software package. Flow cytometric analysis (BD FACSArray Bioanalyzer System) was carried out with a single-cell suspension, and only the live cells were gated based on forward and side scatter dot plots.

### Example 14

### MTT Assay of Self-Assembled, EPIR-Loaded Nanoparticles

MTT assay of the EPIR-loaded biopolymers and nanoparticles was performed using an UT-6100 Microplate Reader.

Approximately 10 000 HeLa cells/well were plated in 96-well plate. The cells were incubated at 37° C. for 24 h. After that the cells were treated with the drug-loaded systems, and incubated at 37° C. for a 72 h. 20 μl MTT reagent was added to each well, and the plate was incubated for 4 h at 37° C. when purple precipitate was clearly visible under microscope, 200 μl DMSO was added to all wells, including control wells. The absorbance of the wells was measured at 492 nm.

### Example 15

### Effect of Glucose Solution on the Size and Polydispersity of Nanoparticles Through a Specific Example

Formulation of a nanoparticle (NP):

mixing PGA-PEG-FA-EPIR (pH=8.0) and CH-EDTA (pH=4) in a ratio of 2PA:1PC, c_{polymer}=0.3 mg/ml

The nanoparticle is mixed with a 75% glucose solution in a ratio so that the final glucose concentration is 5%.

| | |
|---|---|
| Size of the original NP-EPIR(nm) | 117 |
| Polydispersity of the original NP-EPIR | 0.153 |
| Size of NP-EPIR mixed with glucose | 127 |
| solution (nm) | |
| Polydispersity of NP-EPIR mixed with | 0.153 |
| glucose solution | |

### In Vivo Results

Tumor was induced in mice by implanting SK-OV-3 human ovary adenocarcinoma cells s.c. in upper region of back of SCID mice and allowing the tumors to develop to appreciable size over 24 days (70 mm3). The comparative efficacy study of six i.v. injection (day 24, 31, 38, 44, 51 and 58) of 5% glucose, epirubicin (EPIR) 1.8 mg/kg and NP-EPIR (1.8 mg/kg) was evaluated over 72 days. In this table there are: change in tumor volume of mice on 69th day after tumor inoculation (data represent mean %±SEM of five mice per group), change in body weight of mice on 69th day after tumor inoculation (Data represent mean±STDEV of five mice per group) and survival proportion at the end of the experiment.

TABLE 1
Comparative efficacy study in SK-OV-3 s.c.
xenograft SCID mouse model of ovary cancer.

| | Change in | Change in body | Survival |
|---|---|---|---|
| Treatment (total | tumor volume | weight during the | proportion at |
| dose of 6 | (control: | treatment (weight | the end of the |
| injections) | 100%) | at start: 100%) | experiment |
| Control: 5% glucose | 100% ± 30% | 97% ± 7% | 0% |
| EPIR (1.8 mg/kg) | 74% ± 4% | 99% ± 5% | 100% |
| NP-EPIR | 66% ± 3% | 97% ± 6% | 100% |
| (1.8 mg/kg) | | | |

FIG. 1 shows the size distribution of epirubicin-loaded nanoparticles by volume in which nanocarriers were constructed by self-assembly of biopolymers at a concentration of 0.3 mg/ml, at given ratios, where the CH-EDTA solution was added into the PGA-FA-EPIR solution.

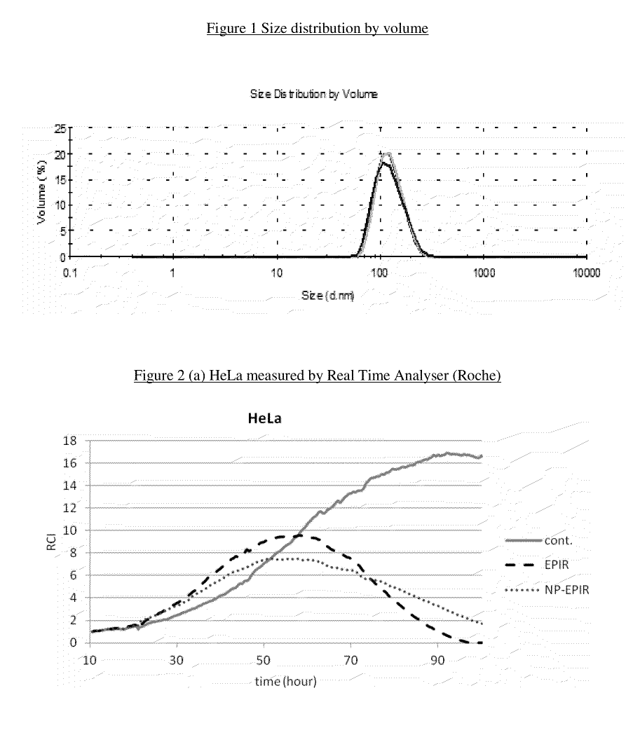

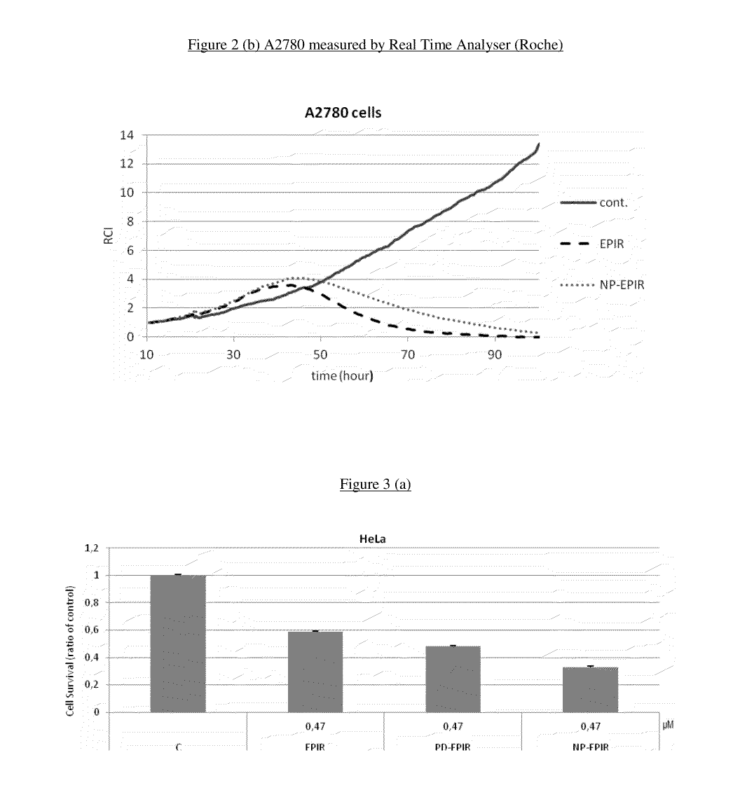

FIG. 2 shows the growth profile of HeLa cells (a) and A2780 cells (b) after treating with epirubicin drug molecules (EPIR), epirubicin-loaded nanoparticles (NP-EPIR), and control cells (C) The injected volume contained the same concentration of epirubicin.

The results show that the effect of epirubicin and epirubicin-loaded nanoparticles is similar on the studied tumor cell lines; however nanoparticles due to their targeting ligands, deliver the drug molecules specifically into the tumor cells and minimize the side effect of the drug. Effect of drug was studied for several days. The results support that effect of drug is long-drawn, the living cell index did not increased even after 3 days.

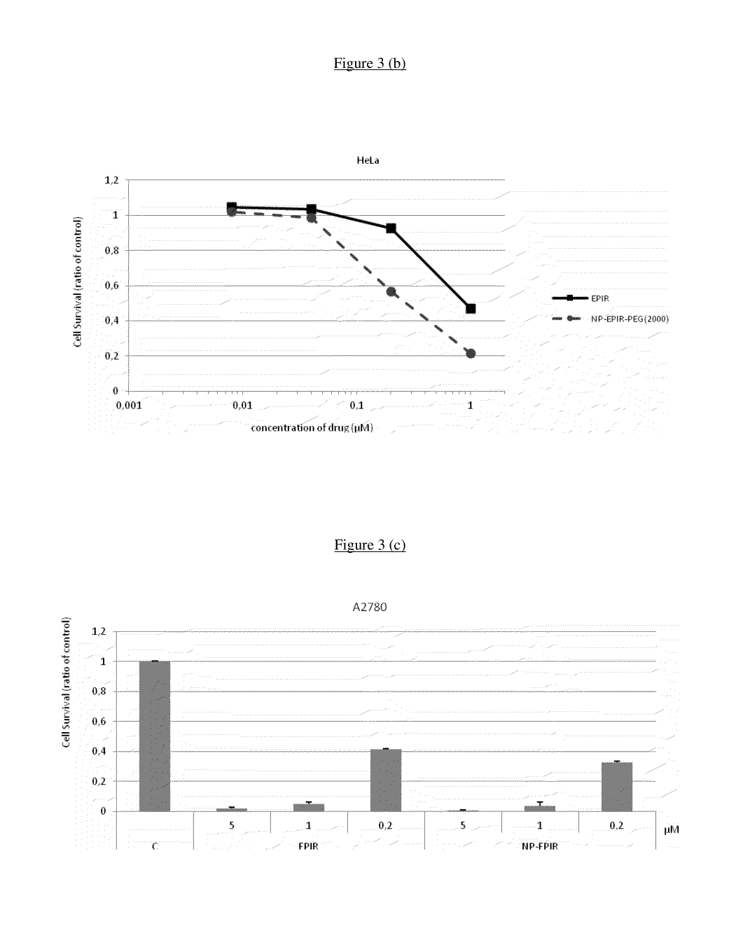

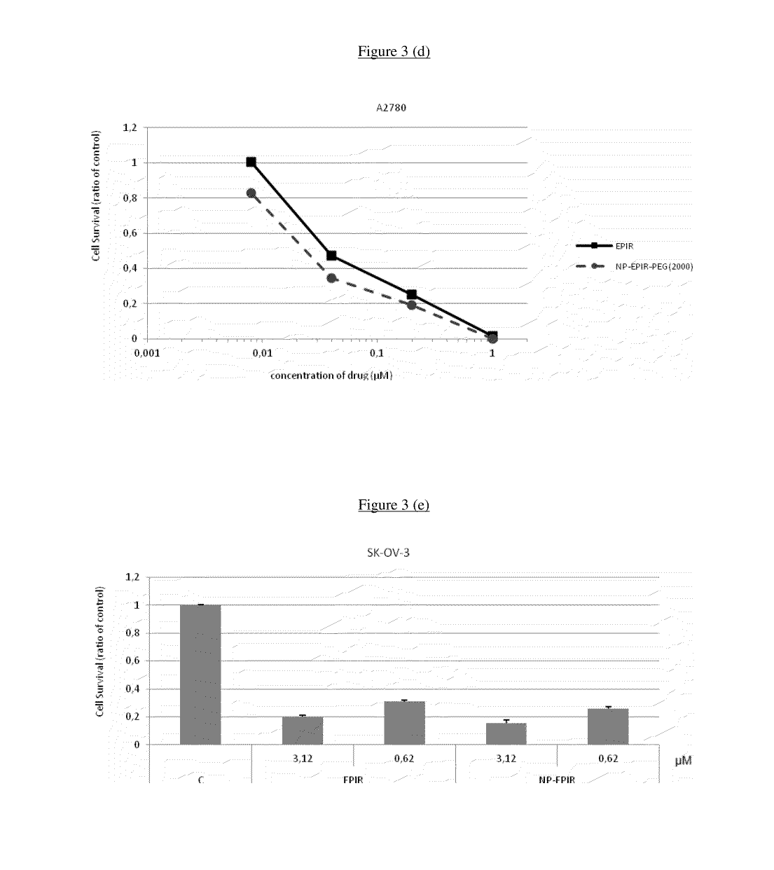

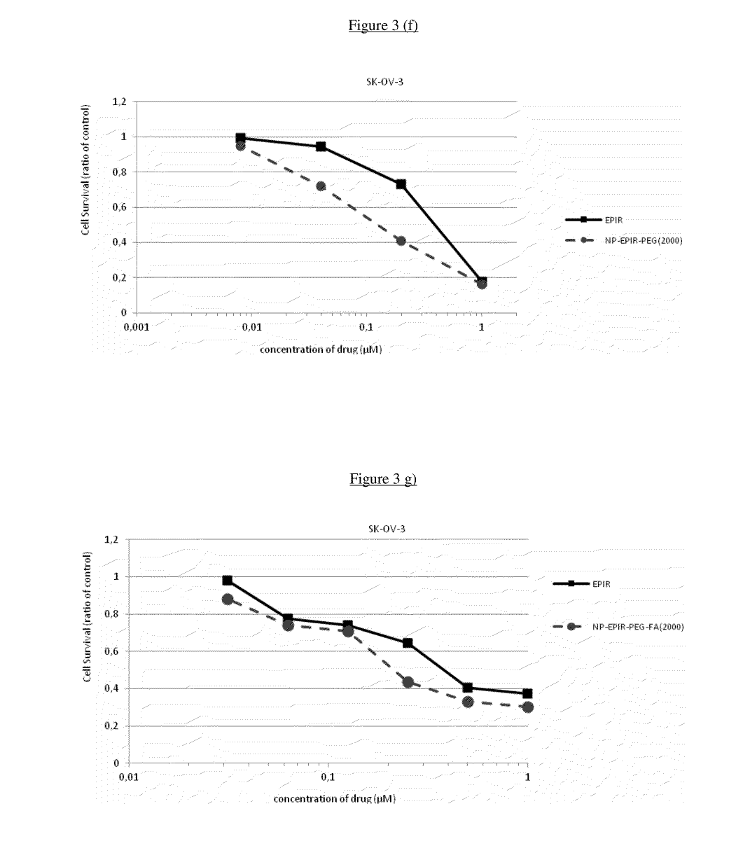

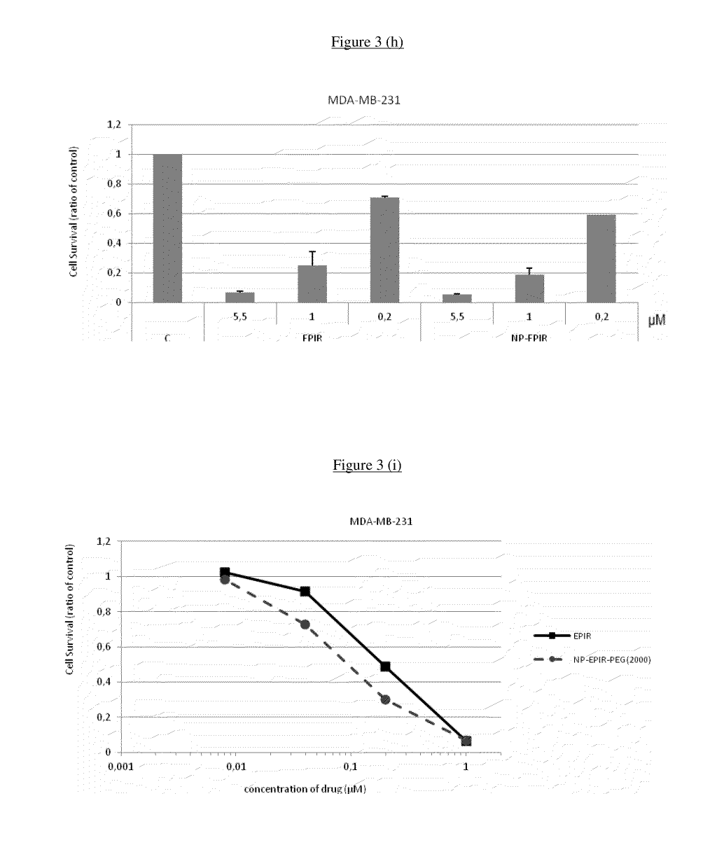

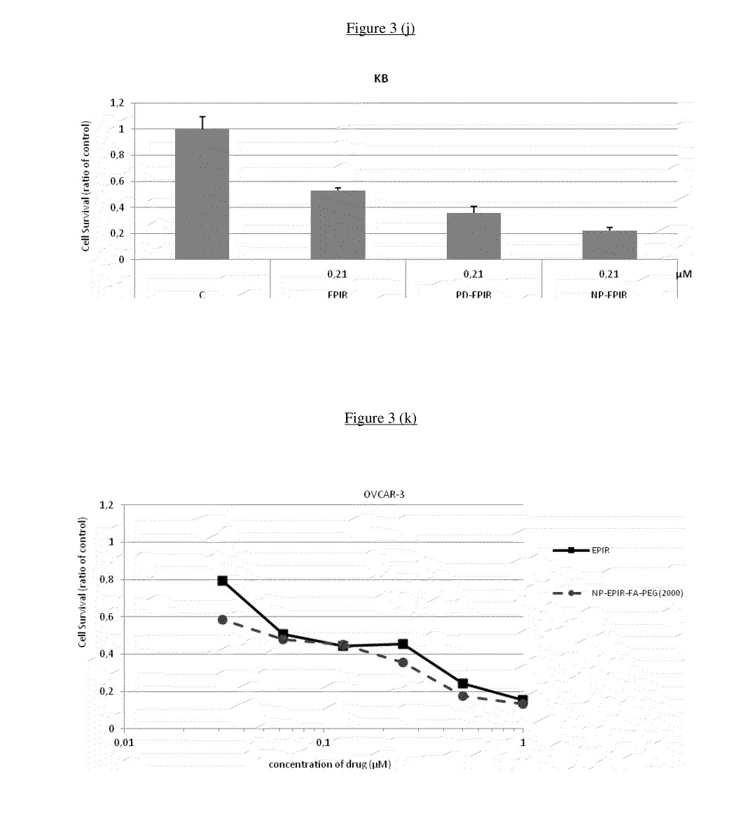

FIG. 3 shows the MTT assay results of epirubicin drug molecules (EPIR) epirubicin-loaded PGA (PD-EPIR) epirubicin-loaded nanoparticles (NP-EPIR), pegylated nanoparticles (NP-EPIR-PEG(2000)) and FA-pegylated nanoparticles (NP-EPIR-PEG-FA(2000)) using HeLa cell line (a,b), A2780 cell line (c,d) SK-OV-3 cell line (e,f,g) MDA-MB-231 cell line (h,i) KB cell line (j) and OVCAR-3 cell line (k).

Results of the MTT assay confirm that the epirubicin was successfully conjugated and the epirubicin-loaded nanoparticles decreased the cell viability of several tumor cells considerably. The viability of tumor cells was investigated in a function of dose of drug-loaded nanoparticles.

1. A stable self-assembled composition comprising
(i) a carrier and targeting system comprising an optionally modified polyanion, and optionally a polycation, which may also be modified; at least one targeting agent which is linked to either the polycation/modified polycation or the polyanion/modified polyanion, or both, or to the surface of the nanoparticle;
(ii) an active compound selected from the group of epirubicin and its pharmaceutically acceptable salts, especially hydrochloride; and optionally
(iii) at least one complexing agent, metal ion, a stabilizer/formulating agent or a PEGylating agent.

2. The composition according to claim 1, wherein the polycation is chitosan, the modified polycation is selected from the group of chitosan-EDTA, chitosan-DOTA, chitosan-DTPA, chitosan-FA, chitosan-LHRH, chitosan-RGD CH-EDTA_FA, CH-FA-EDTA, CH-DOTA-FA, CH-FA-DOTA, CH-DTPA-FA, CH-FA-DTPA; the polyanion is selected from the group of poly-gamma-glutamic acid (PGA), polyacrylic acid (PAA), hyaluronic acid (HA), alginic acid (ALG); the modified polyanion is selected from the group of PGA-EPIR, PGA-FA, PGA-FA-EPIR, PGA-LHRH, PGA-RGD, PAA-FA, PAA-LHRH, PAA-RGD, HA-FA, HA-RGD, HA-LHRH, ALG-FA, ALG-LHRH, ALG-RGD; the targeting agent is selected from the group of folic acid (FA), LHRH, RGD, a monoclonal antibody, preferably Transtuzumab; the complexing agent is selected from the group of diethylenetriaminepentaacetic acid (DTPA), 1,4,7,10-tetracyclododecane-N,—N′,N″,N′″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethylether)N,N,N′,N′,-tetraacetic acid (EGTA), 1,4,8,11-tetraazacyclotradecane-N,N′,N″,N′″-tetraacetic acid (TETA), and 1,4,7-triazacyclononane-N,N′,N″-triacetic acid (NOTA); the derivatives of biopolymers can be their cross-linked nanosystems, biopolymer-complexone products, or other grafted derivatives resulted in modifications of biopolymers with other molecules, e.g. PEG oligomers; the formulating agent is selected from the group of glucose, physiological salt solution, PBS; and the metal ion is selected from the group of calcium, magnesium, copper, gadolinium, gallium.

3. The composition according to claim 1, which is characterized by any or more of the following features:
(i) the average size of the nanoparticles is in the range between 30 to 500 nm, preferably 60 to 200 nm, more preferably about 80 to 120 nm;
(ii) the proportion of the polycation to the polyanion is about 1:20 to 20:1 based on the weight of the agents;
(iii) the polyanion has a pH of 7.5 to 10; a molecular weight of 10 000 Da to 1.5 MDa and a concentration of 0.01 to 2 mg/ml;
(iv) the polycation has a pH of 3.5 to 6; a molecular weight of 60 to 320 kDa and a concentration of 0.01 to 2 mg/ml.

4. A process for the preparation of the composition according to claim 1, characterized in that it comprises the steps of
(i) a targeting agent is bound covalently to the polyanion;
(ii) the active agent is bound covalently or by an ionic bond to the polyanion;
(iii) the polycation and the polyanion are contacted with each other, preferably in a ratio of 1:20 to 20:1 based on the weight of the agents, thus are reacted with each other to self-assemble;
(iv) optionally other components are added to the reaction mixture.

5. The process according to claim 5, wherein the polyanion used has a pH of 7.5 to 10; a molecular weight of 10 000 Da to 1.5 MDa and a concentration of 0.01 to 2 mg/ml; and the polycation used has a pH of 3.5 to 6; a molecular weight of 60 to 320 kDa and a concentration of 0.01 to 2 mg/ml.

6. A stable self-assembled composition comprising
(i) a carrier and targeting system comprising an optionally modified polyanion, and optionally a polycation, which may also be modified; at least one targeting agent which is linked to either the polycation/modified polycation or the polyanion/modified polyanion, or both, or to the surface of the nanoparticle;
(ii) an active compound selected from the group of epirubicin and its pharmaceutically acceptable salts, especially hydrochloride; and optionally
(iii) at least one complexing agent, metal ion, a stabilizer/formulating agent or a PEGylating agent, which is obtainable by the process according to claim 4.

7. A pharmaceutical composition comprising the composition according to claim 1 together with pharmaceutically acceptable auxiliary materials.

8. (canceled)

9. (canceled)

10. A method for the treatment of a subject in need for the treatment of tumours, especially human cervical carcinoma (HeLa, KB), human ovary carcinoma (A2780, SK-OV-3, OVCAR-3), human lung adenocarcinoma (A549, H1975), human breast carcinoma (MCF-7, MDA-MB-231), human prostate carcinoma (PC-3, LNCaP), human skin melanoma (HT168-M1/9), human colon adenocarcinoma (HT29), human melanoma (WM983A) and human metastatic melanoma (WM983B) by administering to the subject an effective amount of the composition according to claim 1.
